(19) United States
(12) Patent Application Publication
(10) Pub. No.: US 2025/0145548 A1
(43) Pub. Date: May 8, 2025

(54) HYBRID CATALYSTS AND METHODS AND SYSTEMS OF CATALYTIC CONVERSION OF CO2 TO C2 COMPOUNDS

(71) Applicants: University of Virginia Patent Foundation, Charlottesville, VA (US); Iowa State University Research Foundation, Inc., Ames, IA (US)

(72) Inventors: Sen Zhang, Crozet, VA (US); Zhouyang Yin, Fremont, CA (US); Long Qi, Ames, IA (US); Wenyu Huang, Ames, IA (US); Jiaqi Yu, Evanston, IL (US)

(21) Appl. No.: 18/817,775

(22) Filed: Aug. 28, 2024

Related U.S. Application Data
(60) Provisional application No. 63/595,879, filed on Nov. 3, 2023.

(51) Int. Cl. C07C 1/12; B01J 23/72; B01J 27/24; B01J 35/45; B01J 37/04

(52) U.S. Cl. CPC C07C 1/12; B01J 23/72; B01J 27/24; B01J 35/45; B01J 37/04; C07C 2531/28

(57) ABSTRACT

The present disclosure provides for hybrid catalysts, methods of converting CO₂ to C2 products (e.g., ethylene), systems for converting CO₂ to C2 products (e.g., ethylene), and the like. The hybrid catalyst of the present disclosure effectively uses two steps of converting CO₂ to ethylene in a single hybrid catalyst.

### CROSS-REFERENCE TO RELATED APPLICATIONS

This application claims the benefit of U.S. Provisional Application entitled “HYBRID CATALYSTS AND METHODS AND SYSTEMS OF CATALYTIC CONVERSION OF CO₂ TO C2 COMPOUNDS” and having Ser. No. 63/595,879, filed Nov. 3, 2023, which is herein incorporated by reference in its entirety.

### FEDERAL SPONSORSHIP

This invention was made with government support under Grant Nos. CBET-2004808 and CHE-2145220, awarded by the U.S. National Science Foundation, and grant DE-AC02-07CH11358, awarded by the Department of Energy. The government has certain rights in the invention.

### BACKGROUND

Reducing CO₂ emissions is of the upmost importance and many resources are directed to reduce CO₂ emission. Utilization of CO₂ as an alternative carbon feedstock for commodity chemical production is a promising strategy to reduce the dependence on fossil fuel resources. While many approaches are being pursued, there is still a need to provide a technology to utilize CO₂ as a carbon feedstock.

### SUMMARY

The present disclosure provides for hybrid catalysts, methods of converting CO₂ to C2 products, systems for converting CO₂ to C2 products, and the like.

The present disclosure provides for a hybrid catalyst comprising a plurality of Cu nanowires and a single-atom nickel on nitrogen assembly carbon (Ni-NAC). In an aspect, the Cu nanowire has a diameter of about 10 to 100 nanometer or 10 to 100 nanometer and a length of about 0.5 to 50 μm or 0.5 to 50 μm, the Ni-NAC is a nitrogen-doped ordered mesoporous carbon embedded with single-atom nickel, wherein the Ni-NAC has a Ni loading of about 1.5 to 3 weight %, the ratio of Cu nanowires:Ni-NAC of about 2:1 to 20:1, and/or the Ni-NAC does not include a Ni nanoparticle, wherein the Ni present in the Ni-NAC consists of single-atom Ni. The Cu nanowires can have a dominant {100} surface facets or the Cu nanowires can have a face-centered cubic metallic structure. The catalyst can also include a powered carbon black mixed with the Cu nanowires and Ni-NAC.

The present disclosure provides for a method of converting CO₂ to ethylene comprising: exposing CO₂ to a hybrid catalyst as described above and herein; and forming ethylene.

The present disclosure provides for a system, comprising: a hybrid catalyst as described above and herein, an introduction system to expose CO₂ to the hybrid catalyst, wherein upon introduction of the CO₂ to the hybrid catalyst ethylene is produced.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 is a schematic illustration of modular design of Cu/Ni-NAC hybrid catalyst for tandem catalysis.

FIG. 2A illustrates a STEM HAADF image of Ni-NAC materials showing ordered porous structure (scale bar=100 nm). FIG. 2B illustrates the atomic resolution STEM image of Ni-NAC. Red arrows illustrate single-atom Ni sites (scale bar=5 nm). FIGS. 2C and 2D illustrate SEM images of Cu NWs (c) and Cu/Ni-NAC hybrid catalyst (d) (scale bar=50 μm).

FIG. 3A illustrates nitrogen sorption isotherm analysis of Ni-NAC. FIG. 3B illustrates pore distribution of Ni-NAC. FIGS. 3C and 3D illustrate R-space plots of Cu (c) and Ni (d) K-edge EXFAS of metal foil standard and hybrid catalyst.

FIGS. 4A and 4B illustrate eCO₂RR (a) products distribution and (b) current density of Cu/Ni-NAC hybrid catalysts in different potentials.

FIGS. 5A and 5B illustrate in situ SEIRAS spectra of Cu/Ni-NAC (a) and Cu NWs (b) catalysts under eCO₂RR conditions. FIG. 5C illustrates in situ XANES of Ni K edge of Cu/Ni-NAC eCO₂RR conditions.

FIG. 6 illustrates XANES results of Cu/Ni-NAC under different conditions.

FIG. 7A illustrates eCO₂RR of Cu/Ni-NAC 10:1 in flow cells with different electrolytes. FIG. 7B illustrates products distribution with Cu/Ni-NAC 10:1 and Cu NWs catalysts in flow cells (electrolyte: 10 M KOH). FIG. 7C illustrates eCO₂RR stability test of Cu/Ni-NAC 10:1 in a flow cell (electrolyte: 10 M KOH).

FIG. 8 illustrates a TEM image of Cu NWs with average diameter of 50 nm.

FIG. 9 illustrates XRD patterns of Cu NWs. Triangles indicate standard Cu pattern.

FIG. 10 illustrates an HRTEM image of Cu NWs.

FIG. 11 illustrates a XRD pattern of Ni-NAC. Blue Ni-NAC pattern, black standard Ni pattern.

FIG. 12 illustrates a typical GC-TCD/FID chromatograms of eCO₂RR with Cu/Ni-NAC as the catalyst.

FIG. 13 illustrates a typical solution ¹NMR spectrum of eCO₂RR with Cu/Ni-NAC as the catalyst.

FIG. 14 illustrates an in situ SEIRAS results of Ni-NAC during eCO₂RR.

FIG. 15 illustrates a flow cell device for eCO₂RR.

FIG. 16 illustrates an eCO₂RR with Ni-NAC in flow cells (electrolyte: 10M KOH).

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

The present disclosure provides for hybrid catalysts and methods and systems for converting CO₂ to C2 (two carbon) compounds (e.g., ethylene).

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, inorganic chemistry, synthetic chemistry, and the like, which are within the skill of the art. Such techniques are explained fully in the literature.

The following description and examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in bar or psig. Standard temperature and pressure are defined as 25° C. and 1 bar.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible. Different stereochemistry is also possible, such as products of cis or trans orientation around a carbon-carbon double bond or syn or anti addition could be both possible even if only one is drawn in an embodiment.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### General Discussion

The present disclosure provides for hybrid catalysts, methods of converting CO₂ to C2 products (e.g., ethylene), systems for converting CO₂ to C2 products (e.g., ethylene), and the like. The hybrid catalyst of the present disclosure effectively uses two steps of converting CO₂ to ethylene in a single hybrid catalyst.

In an aspect, the present disclosure provides for a hybrid catalyst with integrated single-atom Ni (that convert CO₂ to CO) and nanoscale Cu catalytic components (e.g., Cu nanoparticles such as Cu nanowire or Cu plates) that enhances the C—C coupling and C2 product (e.g., ethylene (C₂H₄)) production efficiency in the electrocatalytic CO₂ reduction reaction (eCO₂RR). In an embodiment, the single-atom Ni anchored on high-surface-area ordered mesoporous carbon enables high-rate and selective conversion of CO₂ to CO in a wide potential range, which complements the subsequent CO enrichment on Cu component (e.g., Cu nanowires (NWs)) for the C—C coupling to C₂H₄. In situ surface-enhanced infrared absorption spectroscopy (SEIRAS) confirms the substantially improved CO enrichment on Cu once its integration with single-atom Ni. Also, in situ X-ray absorption near edge structure (XANES) demonstrates the structural stability of the hybrid catalyst during eCO₂RR. By modulating hybrid compositions, the optimized catalyst shows 66% Faradaic efficiency (FE) in an alkaline flow cell with over 100 mA·cm⁻² at −0.5 V vs. reversible hydrogen electrode, leading to a five-order enhancement in C₂H₄ selectivity compared with single-component Cu NWs. Additional details are provided in Example 1.

The present disclosure provides for a copper/single-atom nickel on nitrogen assembly carbon (Cu/Ni-NAC) hybrid catalyst that includes a plurality of Cu components (e.g., Cu nanowires). In an embodiment, the Cu component can be Cu nanoparticles or Cu plates. In an embodiment, the Cu component is Cu nanowire, which is described in detail in Example 1. In an aspect, the Cu/Ni-NAC hybrid catalyst can have a ratio of Cu nanowires:Ni-NAC of about 2:1 to 20:1, about 2:1 to 10:1, about 5:1 or 10:1. The Cu/Ni-NAC hybrid catalyst can also include a powered carbon black mixed with the Cu nanowires and Ni-NAC. The Cu/Ni-NAC hybrid catalyst can have a ratio of Cu nanowires:Ni-NAC:powered carbon black of about 2:1:1 to 10:1:9.

In an aspect, the Cu nanowires can have a diameter about 10 to 100 nm and a length of about 0.5 to 50 μm. The Cu nanowires can have a dominant {100} surface facets. The Cu nanowires can have a face-centered cubic metallic structure. The Cu nanowires can facilitate the CO-to-ethylene conversion.

In an aspect, the Cu nanoplate (or nanosheet) can have length and/or width of about 1-2 μm and having an exposed (111) surface. The Cu nanoplate can facilitate the CO-to-acetate conversion. The Cu nanoplate can be those as described in Nature Catalysis volume 2, pages 423-430 (2019), which is incorporated herein by reference.

In an aspect, the nanoparticles can have a diameter of about 20-30 nm and can have abundant grain boundaries. The Cu nanoparticles can facilitate the CO-to-ethanol conversion. The Cu nanoparticles can be those as described in ACS Cent. Sci. 2016, 2, 3, 169-174, which is incorporated herein by reference.

In an aspect, the Ni-NAC can be a nitrogen-doped ordered mesoporous (e.g., about 2.5 to 15 nm in pore size) carbon embedded with single-atom nitrogen. The Ni-NAC can have a surface area of about 760 to 995 m²/g. In an aspect, the Ni-NAC does not include a Ni nanoparticle. The Ni-NAC has a Ni loading of about 1.5 to 3 weight %. Additional details are described in reference 42, which is included herein by reference.

Aspects of the present disclosure provide for methods of forming a C2 product (e.g., ethylene) from CO₂ using the hybrid catalyst described herein. The product formed can be determined based on the Cu component. For example, ethylene can be formed if Cu nanowires are used as the Cu component. Acetate can be formed if the Cu component is a Cu nanoplate. Ethanol can be formed if the Cu component is copper multigrain nanoparticles. In an aspect, the Cu component is a Cu nanowire. Additional details regarding the use of Cu nanowires in the hybrid catalyst are provided in Example 1.

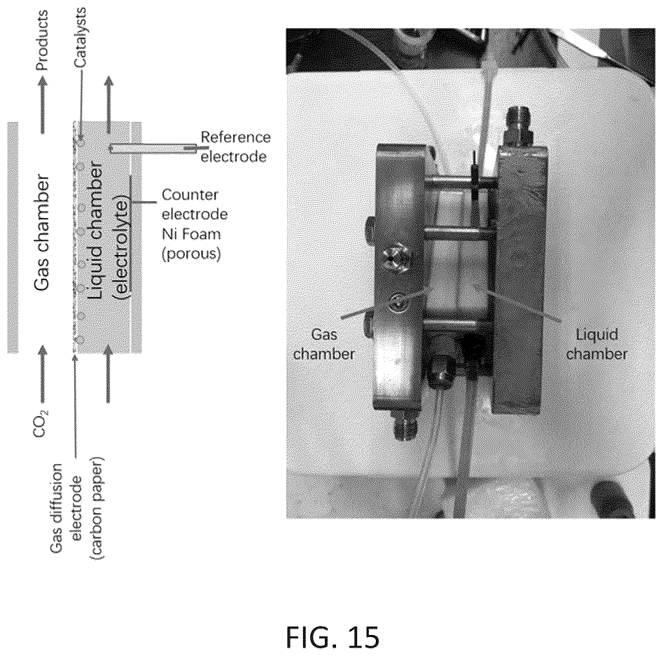

In an embodiment, the method can include exposing CO₂ to a Cu/Ni-NAC (Cu nanowire in this example) hybrid catalyst and CO₂ reacts with the Cu/Ni-NAC hybrid catalyst to produce ethylene. The CO₂ can be exposed using a flow of CO₂ across the hybrid catalyst surface or in a batch mode where CO₂ is flowed into a chamber that includes the hybrid catalyst. The amount of CO₂ that can be processed into ethylene can depend upon the surface area of the hybrid catalyst and the other electrochemical variables as well as if a flow or batch mode is utilized. In an aspect, the method can produce ethylene with 50% Faradaic efficiency (FE) at −1.6 V vs. RHE in 0.5 M KHCO₃ solution and with 66% FE at −0.5 V vs. RHE in 10 M KOH condition, which is described in more detail in Example 1. FIG. 15 in Example 1 illustrates a system that can be used for performing the method.

In general, the system includes positioning the Cu/Ni-NAC hybrid catalyst in a closed system and exposing (e.g., flowing) CO₂ across the Cu/Ni-NAC hybrid catalyst, where a reaction with CO₂ and the hybrid catalyst occurs and the C2 product (e.g., ethylene) is formed. FIG. 15 in Example 1 illustrates a system that can be used to form the C2 product. For example, a system can include two chambers, the first chamber including CO₂ and the second chamber including the electrolyte (e.g., KOH, or KHCO₃). The first chamber and the second chamber are separated by a working electrode that includes the hybrid catalyst (present on the electrolyte side of the working electrode) of the present disclosure. A counter electrode such as Ni foam can be used. The two-step conversion of CO₂ to ethylene (if Cu nanowires are used) can then be performed (details are provided in Example 1).

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

Reaching net-zero CO₂ emission by 2055 is critical to limiting global warming to 1.5° C. above the pre-industrial periods, as emphasized by the Intergovernmental Panel on Climate Change (IPCC).¹ The utilization of CO₂ as an alternative C₁ feedstock for commodity chemical production is a promising strategy to reduce and even eliminate the dependence on fossil fuel resources for a rapid decarbonization.^{2,3} Compared with conventional thermal conversion of CO₂, eCO₂RR is advantageous in terms of prospects for distributed chemical manufacturing under ambient conditions, and the direct use of clean electrical energy and the proton source in water.⁴⁻⁷ In the past few decades, eCO₂RR has been extensively investigated, and among various catalytic materials studied, Cu is the most appealing one due to its ability to enable C—C coupling to produce C₂₊ hydrocarbons and oxygenates that are more valuable than C₁ products (CO, CH₄, formate).⁸⁻¹⁰ Substantial efforts have been devoted to tailoring Cu catalysts to enhance the C²⁺ production, including tuning crystalline plane of Cu (e.g., (100))^{11,12} or boundary/stepped Cu surface,¹³⁻¹⁵ alloying with other elements,^{16,17} adjusting Cu oxidation state,¹⁸⁻²⁰ surface ligand modification,²¹ synthesizing Cu-based metal-organic frameworks,²²⁻²⁵ and modulating catalyst-electrode integration.^{20,26,27} To get more insights of the C—C bonding, a variety of in-situ characterization methods have also been developed.²⁸⁻³³ Despite some progress, Cu-based catalysts still remain low in energy efficiency and product selectivity, especially when targeting one specific C₂₊ product rather than a mixture.^{6,34,35}

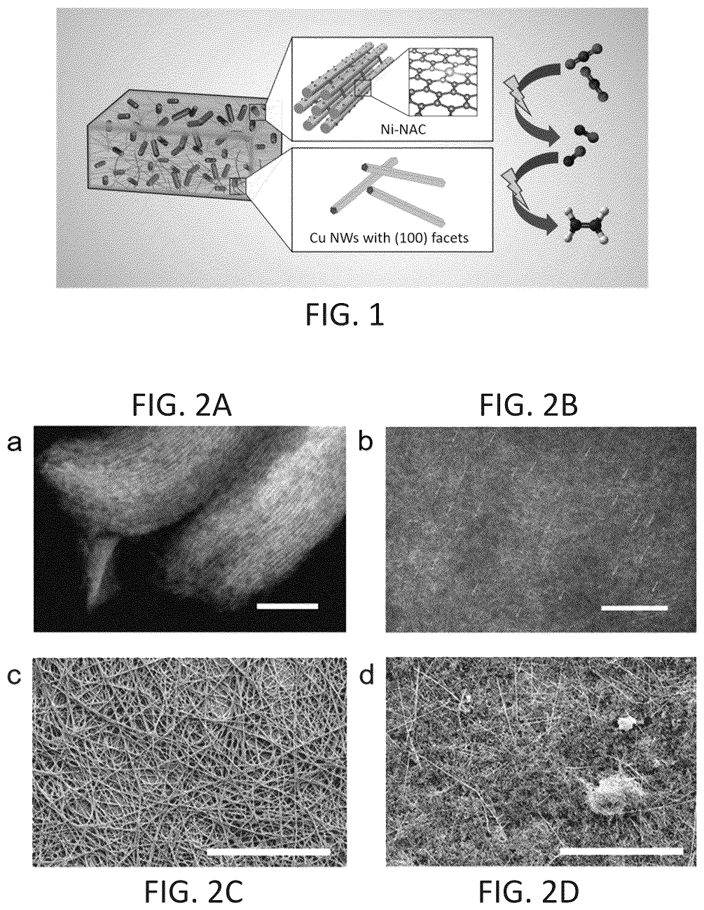

In this example a hybrid catalyst with two functional modules, single-atom Ni and nanoscale Cu being properly coupled to facilitate selective production of C₂H₄ in eCO₂RR (FIG. 1) is described. C₂H₄ is one of the largest volume organic chemicals produced in petrochemical industry, and is broadly used to synthesize plastics and other chemicals.³⁶ Previous theoretical work suggested that the adsorbed *CO is an important intermediate for the subsequent C—C coupling, and experimentally, Cu with the (100) catalytic surface is among such catalysts.³⁷⁻⁴⁰ However, Cu is kinetically sluggish in the CO₂-to-CO step, which limits the surface coverage of *CO and results in high overpotential and low selectivity for C₂H₄ production.⁴¹ In principle, a hybrid catalyst that cascades CO₂-to-CO and CO-to-C₂H₄ conversions should greatly favor the C₂H₄ production, but this requires these two complementary conversion steps to have matching overpotentials for the maximized synergy. Very recently, we have developed a single-atom Ni catalyst anchored on nitrogen assembly carbon (Ni-NAC) that can catalyze CO₂-to-CO with more than 90% Faradaic efficiency (FE) at a wide potential range, due to the enhanced mass transfer from high-surface area ordered porous architecture and the abundant single-atom Ni catalytic centers.^{42,43 (ref 42 is incorporated herin by reference)} We envision that such a single-atom Ni-NAC material is an ideal candidate to complement Cu for the tandem conversion. Our results show that, by properly assembling single-atom Ni-NAC and Cu NWs with dominant {100} surface facets, a high-rate eCO₂RR with a 66% FE to C₂H₄ at moderate potential {−0.5 V vs. reversible hydrogen electrode (RHE)} is obtained. In situ SEIRAS provides direct evidence of substantially enhanced CO enrichment on Cu in the hybrid catalyst. Our work highlights the modular design of hybrid catalysts to optimize reaction kinetics for eCO₂RR and potentially for other complex reactions.

### Results and Discussion

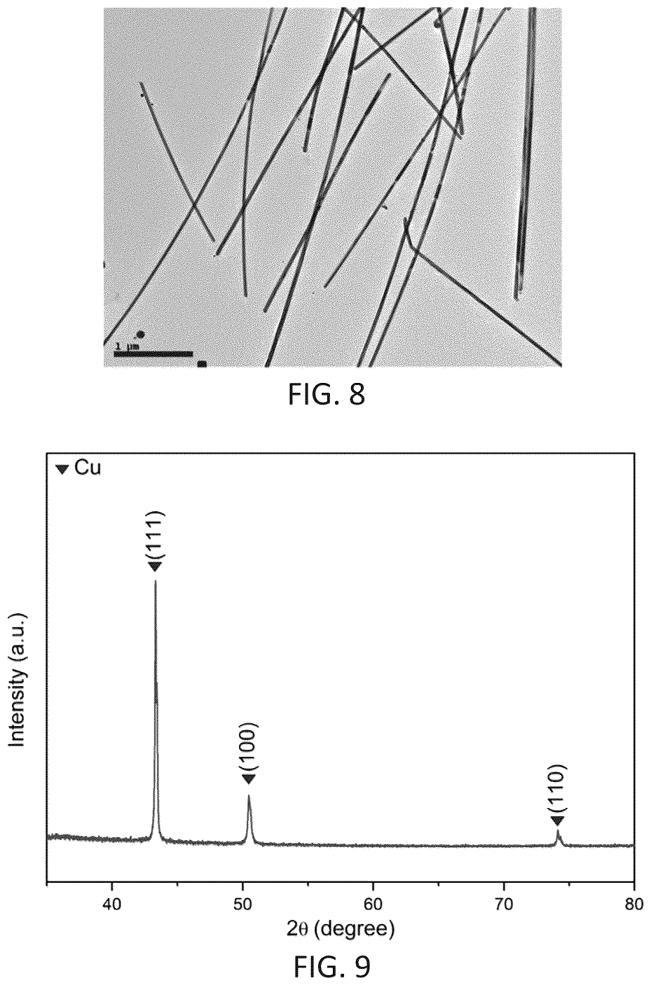

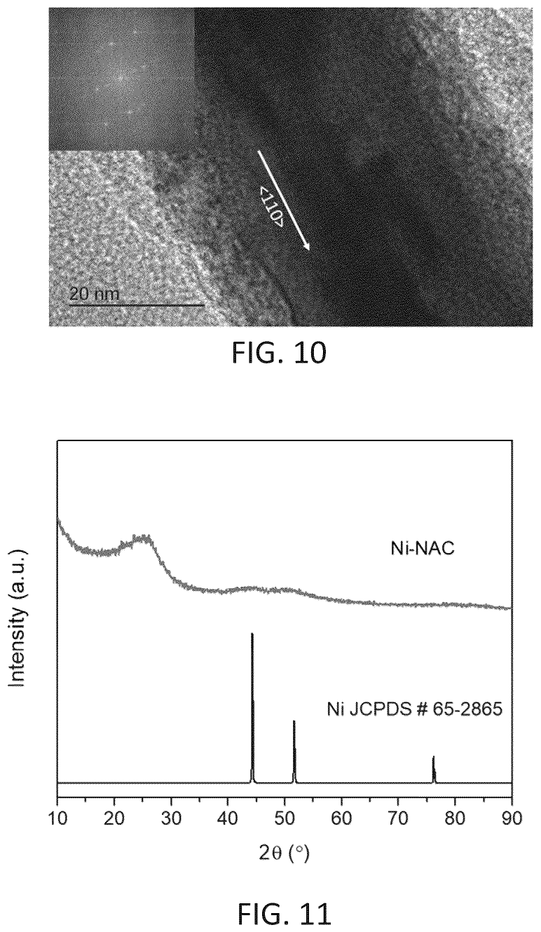

Catalyst Preparation and Characterization. The single-atom Ni-NAC and Cu NWs were synthesized separately, according to the reported methods (Supporting information).^{37,42} As shown in FIG. 8, the as-synthesized Cu NWs have a diameter of ˜50 nm and a length of several to tens of μm. The face-centered cubic (fcc) metallic Cu structure is confirmed using X-ray diffraction (XRD) (FIG. 9). The surface of Cu NWs is mainly composed of {100} facets (FIG. 10), which have been proven to be active for the CO reduction to C₂H₄.^{11,44} The single-atom Ni-NAC possesses an ordered mesoporous architecture, as indicated by the aberration-corrected scanning transmission electron microscopy (STEM) high-angle annular dark field (HAADF) image (FIG. 2a). The formation of single-atom Ni is also visualized in HAADF-STEM image (FIG. 2b). After Cu NWs and Ni-NAC were successfully synthesized, they were mixed in hexanes under sonication to generate the hybrid catalysts (FIG. 1). As shown in the scanning electron microscopy (SEM) image in FIG. 2c, before assembly, high-purity Cu NWs intersect with each other. After assembly (FIG. 2d), the Cu NWs are uniformly embedded in the matrix of Ni-NAC and Vulcan carbon, suggesting that Cu NWs and Ni-NAC are in close contact. In addition, the morphology of Cu NWs is not changed after the formation of the hybrid catalysts.

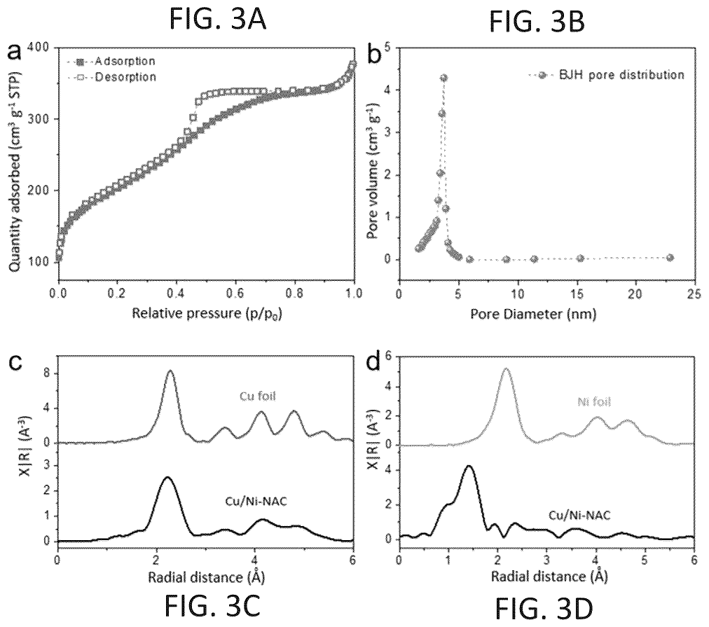

Besides electron microscopy for morphology characterization, N₂ physisorption measurement shows that the surface area of single-atom Ni-NAC sample reaches 718 m² g⁻¹ due to the uniform (FIG. 3a) and interconnected 3.0 nm pores (FIG. 3b). Consistent with our previous report, the Ni-NAC sample presents single-atom Ni on the graphitic N-doped carbon with a Ni loading of 1.7 wt. % while no Ni nanoparticles are formed, as interpreted from the ICP results, the XRD pattern (FIG. 11), and extended X-ray absorption fine structure (EXAFS) measurement (discussed below). To understand the atomical coordination structure of the hybrid material, we performed ex situ EXAFS measurements of Cu/Ni-NAC, as summarized in FIGS. 3c and d. The R-space plot of the hybrid catalyst is consistent with the Cu foil standard, in which a Cu—Cu scattering pathway is observed at 2.2 Å, a typical feature for fcc Cu structure (FIG. 3c). Unlike the EXAFS of Cu, the main peak in the R-space plot of Ni locates at 1.45 Å, which is identical to the Ni—N peak in reported Ni-phthalocyanine (Pc) standard (FIG. 3d).⁴⁵ Compared with the Ni foil reference, there is no detectable Ni—Ni peak in the hybrid catalyst. Therefore, the hybrid catalyst contains both single-atom Ni and continuous Cu metallic structures.

In order to investigate the bi-component effect and avoid CO deficiency or overproduction, Cu NWs and Ni-NAC with different ratios are assembled and evaluated. Cu NWs:Ni-NAC:Vulcan carbon weight ratio are controlled to be 2:1:1 (Cu/Ni-NAC 2:1), 5:1:4 (Cu/Ni-NAC 5:1), and 10:1:9 (Cu/Ni-NAC 10:1), where the eCO₂RR-inert carbon support (Vulcan carbon) is added to maintain the conductivity of the catalysts and the carbon (Vulcan carbon+Ni-NAC)/Cu ratio is unchanged (1:1).

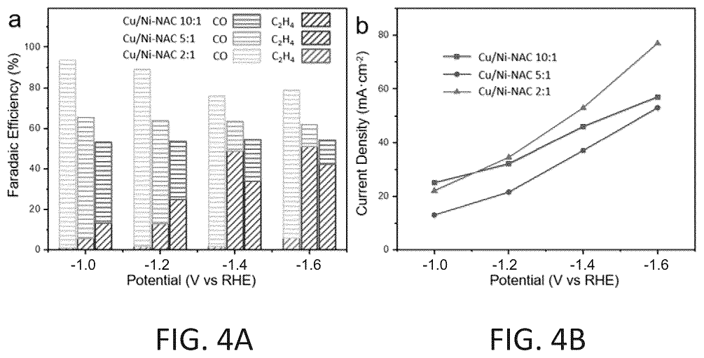

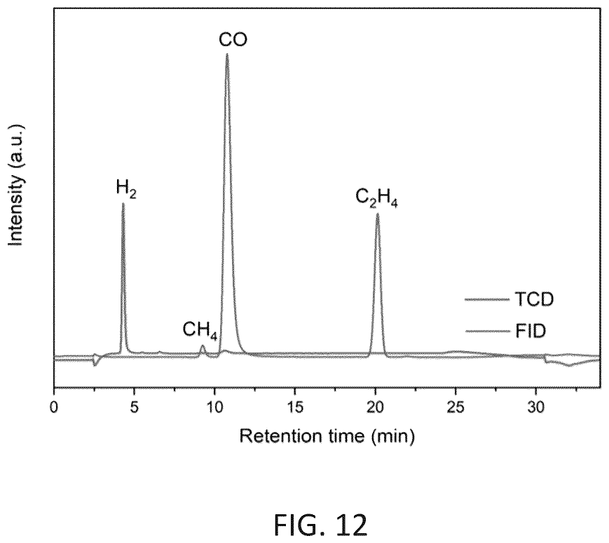

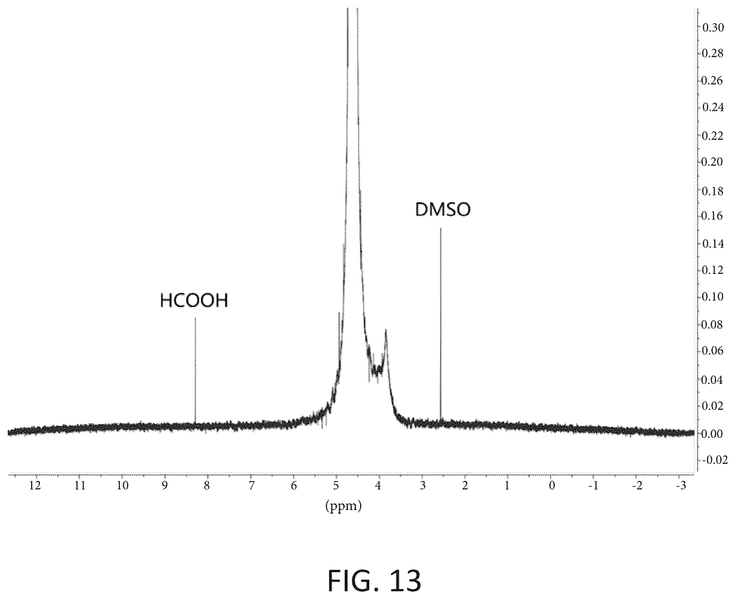

Electrochemical Performance. We first used a H-cell in a neutral electrolyte (0.5 M KHCO₃) to evaluate the impact of different compositions of Cu/Ni-NAC for the eCO₂RR, where online gas chromatograph (GC) and nuclear magnetic resonance (NMR) were employed to analyze gas and liquid products, respectively. The typical GC and NMR spectra are displayed in FIGS. 12 and 13. As summarized in FIG. 4a, the Cu/Ni-NAC 2:1 sample shows the highest FE toward CO among the three hybrid catalysts, indicating that a high ratio of single-atom Ni module leads to CO overproduction. As a result, although the C₂H₄ yield becomes higher as the overpotential increase, the highest C₂H₄ FE for the same catalyst is still less than 10%. We found that increasing the relative content of Cu NWs in the hybrid catalyst significantly enhances C₂H₄ FE. Using the Cu/Ni-NAC 5:1 catalyst, the highest C₂H₄ FE could reach 50% at −1.4 V vs RHE, but only 14% of C₂H₄ is obtained at −1.2 V. With more Cu NWs incorporated, Cu/Ni-NAC 10:1 produces 23% of C₂H₄ at −1.2 V, but has slightly lower FE at higher overpotentials. The current densities of these hybrid catalysts are displayed in FIG. 4b. From −1.2 to −1.6 V, Cu/Ni-NAC 2:1 has the highest current density (up to 80 mA·cm⁻²) due to the highest catalyst loading amount. Although the Cu NWs and Ni-NAC loading are lowered in Cu/Ni-NAC 5:1 and Cu/Ni-NAC 10:1 due to increased use of Vulcan carbon, their current densities are just slightly lower with greatly enhanced C₂H₄ FE. Taking both C₂H₄ FE and current density into consideration, Cu/Ni-NAC 10:1 stands out with the optimized performance and is specifically discussed below for in situ spectroscopy studies and flow-cell testing.

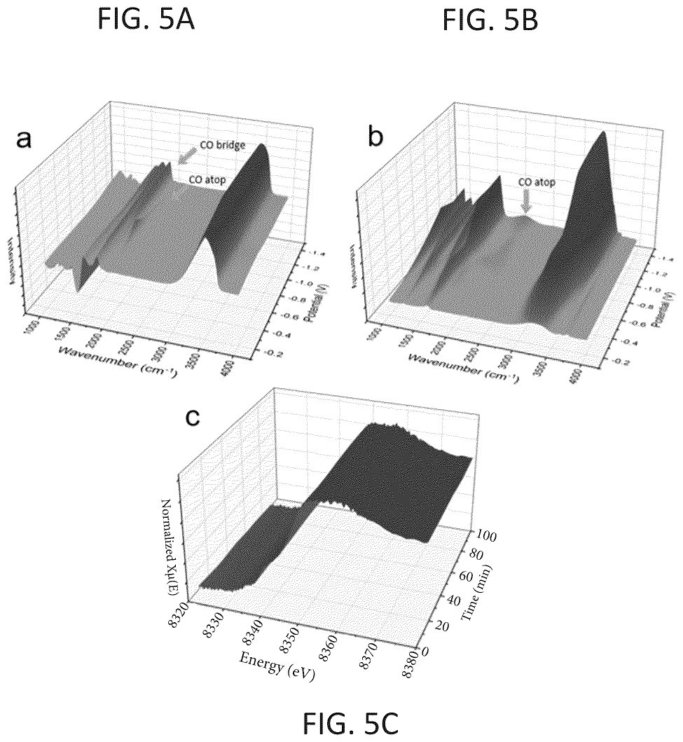

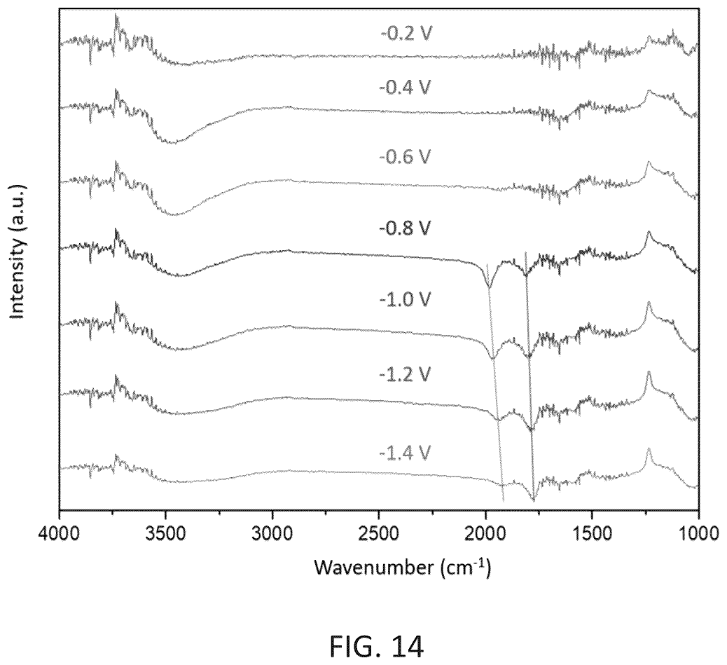

In situ Spectroscopy. To confirm our hypothesized coupling mechanism in hybrid catalysts, we used in situ SEIRAS to elucidate the reaction intermediates and pathways during the eCO₂RR. We deposited a thin film of Au nanoparticles on the silicon attenuated total reflection crystal that serves as both a working electrode and a surface enhancement layer to improve intermediate signals.⁴⁶ Then, our catalysts were uniformly spin-coated on the Au-modified silicon crystal, and we ensured that Au film was fully covered to avoid Au interference in eCO₂RR. As shown in FIG. 5a, the hybrid Cu/Ni-NAC catalyst exhibits two strong CO adsorption peaks at 2050 and 1950 cm⁻¹ during the negative scan, representing clear evidence that *CO generated from Ni-NAC module is transferred and enriched on the surface of Cu NWs for the subsequent C—C coupling. As the potential becomes more negative, the atop-bound *CO band intensity (2050 cm⁻¹) decreases, and the bridge-bound CO peak (1950 cm⁻¹) increases, which is in accordance with previous report.⁴⁷ In contrast, with Cu NWs as the catalyst, there are no peaks associated with bridge-bound CO were observed, and a small atop-bound CO peak only appeared after −0.8 V (FIG. 5b), indicating a low coverage of *CO on Cu NWs during the eCO₂RR. In addition, control experiments with only Ni-NAC catalyst were also tested using in situ SEIRAS under the same condition (FIG. 14), and there is no detectable *CO peak, suggesting that the effective integration of Ni-NAC and Cu NWs are essential for such a strong *CO enrichment phenomenon.

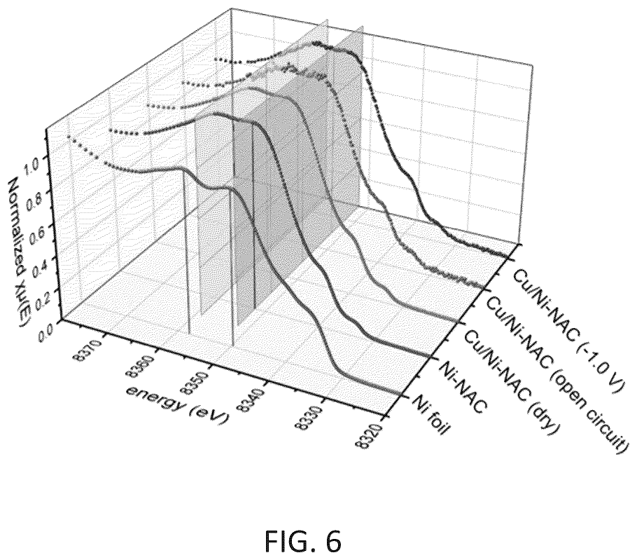

Furthermore, Ni K-edge XANES spectra were also analyzed and summarized in FIG. 6. It is clearly seen that, for both as-prepared and post-electrolysis, ex situ Ni K-edge XANES spectra of the hybrid catalyst are distinctive from Ni foil but identical to Ni-NAC. In addition, under in situ conditions for eCO₂RR, in situ Ni K-edge spectra remain unchanged (FIG. 5c), confirming the high stability of single-atom Ni structures in the hybrid catalyst.

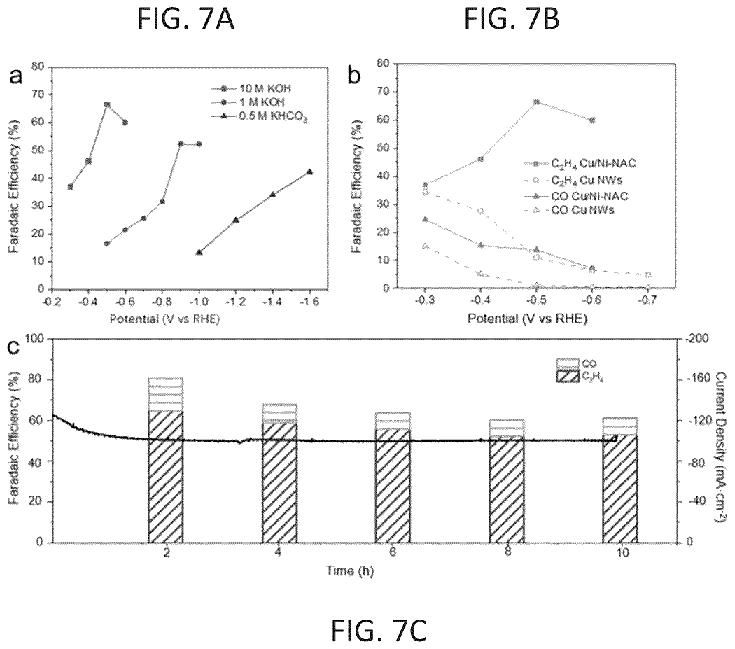

Performance Optimization with Flow Cell. Changing the neutral-pH electrolyte to alkaline solutions should suppress the competing hydrogen evolution reaction (HER) and boost the C—C coupling reaction in eCO₂RR; however, it is likely to cause bicarbonate/carbonate formation and crossover issue in the full electrolyzer. To minimize CO₂ and alkaline solution reaction and to optimize electrolyzer performance, we assembled a flow cell with Cu/Ni-NAC 10:1 catalyst-modified gas diffusion electrode and 1 M or 10 M KOH electrolyte (Figure S8). As shown in FIG. 7a, in 1 M KOH Cu/Ni-NAC 10:1 starts to produce more than 15% of C₂H₄, and the C₂H₄ FE keeps increasing with the overpotential. At −0.9 V, the C₂H₄ FE reaches a plateau of 53%. A more basic condition, 10 M KOH, leads to higher C₂H₄ yield and lower onset overpotential for C₂H₄ production. It starts to generate 37% C₂H₄ at −0.3 V and reaches a peak of 66% C₂H₄ at −0.5 V. Clearly, by controlling electrolyte pH in the flow cell, our hybrid catalyst can deliver a further enhanced C₂H₄ FE from 40% (neutral) to 66% (10 M KOH) at a significantly decreased overpotential.

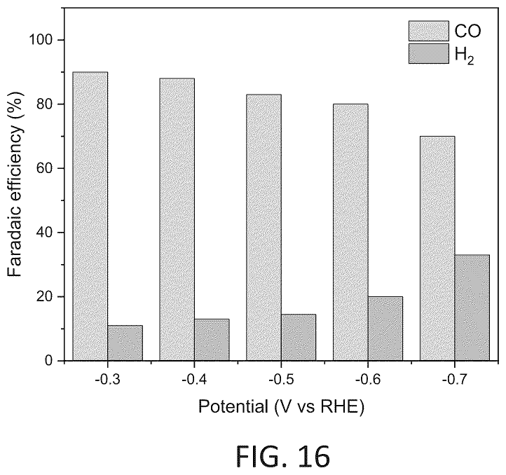

To further demonstrate the synergy of two components in the hybrid catalyst, a control experiment with pure Cu NWs catalyst was carried out using the same flow cell condition (10 M KOH). We observed that Cu NWs behaves similarly as Cu/Ni-NAC 10:1 by enabling 30-40% C₂H₄ FE at −0.3 V (FIG. 7b). However, at more negative overpotentials, the CO formation drops to nearly 0 with Cu NWs because of the significantly higher rate of competitive hydrogen evolution reaction. The lack of CO production leads to a dramatic drop in the C₂H₄ yield with Cu NWs. On the contrary, due to the high CO formation activity of Ni-NAC, the CO formation with the Cu/Ni-NAC catalyst remains higher than 10% at more negative potentials. With abundant CO intermediates, the Cu NWs in close contact with Ni-NAC remains high selectivity towards C₂H₄. In addition, the ability of Ni-NAC to generate sufficient CO within a wide potential range is further demonstrated upon the investigation of the Ni-NAC catalyst for eCO₂RR under the same alkaline conditions (FIG. 16).

Meanwhile, we assessed our hybrid catalyst stability for the eCO₂RR in 10 M KOH (FIG. 7c). After 10 hours of constant potential electrolysis at −0.5 V under the flow cell condition, the C₂H₄ FE drops slightly from 66% to 53%, while the current density remains above 100 mA·cm⁻². Compared with recent Cu-based catalysts for eCO₂RR to ethylene, our hybrid catalyst stands out with both high selectivity and activity at low overpotentials (Table S1). Therefore, Cu/Ni-NAC 10:1 shows promise as a robust catalyst to selectively reduce CO₂ to C₂H₄ at low overpotentials.

### EXPERIMENTAL SECTION

Chemicals. Nickel (II) acetylacetonate (95%), carbon tetrachloride (99.9%), ethylenediamine (99.5%), tetraethyl orthosilicate (TEOS, 98%), and pluronic P123 (Mn˜5,800), oleylamine (OAm, >70%), copper (I) chloride (CuCl, 98%) were purchased from Sigma-Aldrich. Hydrofluoric acid (trace metal grade), hydrochloric acid (trace metal grade), nitric acid (trace metal grade) and N-Methyl-2-pyrrolidone (NMP) were purchased from Fisher Chemical. Platinum wire (0.5 mm diameter, Premion®, 99.997%) and Toray Carbon Paper (TGP-H-60) were obtained from Alfa Aesar. Polyvinylidene fluoride (PVDF) was purchased from MTI corporation. All chemicals were used as received without further purification.

Synthesis of Ni-NAC. Ni-NAC was synthesized according to our previous report.⁴² In a typical synthesis, SBA-15 was obtained first for the template-growth of Ni-NAC. Pluronic P123 was firstly dissolved in hydrochloric solution at 40° C. After being dissolved, TEOS was added and the solution was kept stirring for 2 h. The composition of the achieved gel was: 1 TEOS: 0.017 P123: 5.68 HCl: 197 H₂O. The hydrothermal synthesis was performed in an oven at 100° C. for 24 h in a sealed polypropylene bottle. The white solid material was recovered by Buchner filtration, washed with distilled water, and dried overnight in an oven at 80° C. The obtained solid was then calcined in a muffle furnace at 350° C. under air for 4 h with a heating rate of 2° C. min−1 to produce SBA-15. The Ni acetylacetonate was added into a solution of ethylenediamine (1.80 g) and carbon tetrachloride (4.00 g), followed by the addition of SBA-15 (0.80 g). The mixture was then heated in an oil bath set at 90° C. for 16 h for condensation before the oil bath temperature increased to 120° C. for 4 h to remove the uncondensed ethylenediamine and carbon tetrachloride. The obtained powders were calcined under Ar flow, with temperature raised in a ramping rate of 3° C. min⁻¹ and further maintained at 800° C. for 2 h. The achieved black powder was then etched with 5 wt % HF solution to remove the SBA-15. The catalyst was recovered via centrifuge, washed with deionized water until the pH reached 7. The catalyst was further dried at 100° C. for future usage.

Synthesis of Cu NWs. Cu NWs were synthesized using the reported method.³⁷ 0.1 g of CuCl was dissolved in 3 mL of OAm at 25° C. under Ar flow and vigorous magnetic stirring for 15 min. The solution was then heated to 120° C. for 30 min for degassing. After that, the reaction solution was heated to 180° C. at 20° C. min⁻¹ and kept at this temperature for 90 min. After cooled down to room temperature, 40 mL of hexane was added to collect 50 nm wide Cu NWs via centrifugation (1000 rpm, 3 min). The product was purified by adding 40 mL of hexane and centrifugation for 3 times and then re-dispersed in hexane.

Synthesis Cu/Ni-NAC hybrid catalysts. The as-prepared Cu NWs (dissolved in hexane), Ni-NAC, and KJ Carbon are mixed in certain ratio and sonicate in hexane for 2 hours. The product was purified by adding 40 mL of hexane and centrifugation for 3 times and then dried in N₂ gas.

Characterization. Scanning electron microscope (SEM) imaging was obtained using the FEI Quanta 650 operated at 10 kV. Aberration corrected scanning transmission electron microscope (STEM) imaging was performed using the FEI Titan Themis. Powder X-ray diffraction (XRD) was carried out on a Bruker D8A25 diffractometer with Cu Kα radiation (λ=1.54184 Å). N₂ physisorption was performed using an auto-adsorption analyzer (Micromeritics, 3Flex) at −196° C. Inductively coupled plasma mass spectroscopy (ICP-MS) analysis for metal loadings was performed using Agilent 7700 ICP-MS. SEIRAS are collected by Thermo Scientific Nicolet iS50 FTIR Spectrometer. X-ray absorption spectroscopy (XAS) of the catalysts were measured at Beamline 9-BM of the Advanced Photon Source in Argonne National Laboratory. The in situ XANES spectra of Ni K-edges were collected at beamline 7-BM of the National Synchrotron Light Source II (NSLS-II) at Brookhaven National Laboratory. Details about the in situ XAS experiments can be referred to Ref. 48.

Electrode Preparation. 40 mg of the dried catalyst powder was ground with 4 mg of PVDF with a few drops of NMP to produce catalyst paste that was painted directly onto a 1.0 cm×2.0 cm carbon paper. The catalyst-decorated carbon paper was dried in a vacuum-oven overnight and served as a working electrode.

eCO₂RR Test in H-cell. Autolab electrochemical potentiostat was used to conduct eCO₂RR experiments in aqueous 0.5 M KHCO₃. A platinum wire was used as the counter electrode. All potentials were measured against an Ag/AgCl reference electrode (4.0 M KCl, Pine instrument) and were converted to those against RHE. The experiments were performed in a gas-tight cell with two compartments separated by an anion exchange membrane (Nafion® 212). Each compartment contained 12 mL of electrolyte with approximately 10 mL of headspace. The deionized water was obtained from a Millipore Autopure System.

eCO₂RR Test in Flow Cell. Flow cell used for eCO₂RR is shown in Figure S8. A 2 cm×2 cm Ni foam was used as the counter electrode. All potentials were converted to those against RHE. The experiments were performed in a gas-tight cell with two compartment separated by catalyst-loaded carbon paper/working electrode (the catalyst is loaded on the liquid phase side). The gas phase compartment is flowed by 10 sccm of CO₂ gas and the liquid phase compartment is filled by circulated 25 mL of electrolyte.

Product Analysis. Before the experiment, the electrolyte in the cathode compartment was saturated with CO₂ by bubbling CO₂ gas for at least 30 min and was stirred at 900 rpm. CO₂ gas was delivered at an average rate of 10 sccm (at room temperature and ambient pressure) and routed directly into the gas sampling loop of a gas chromatograph (SHIMADZU GC-2014). The gas phase composition was analyzed by GC every 35 min. The GC analysis was set up to split the gas sample into two aliquots whereof one aliquot was equipped with a thermal conductivity detector (TCD) for H₂ quantification. The second aliquot was equipped with a methanizer and a flame ionization detector (FID) for analyzing CO and C1 to C3 hydrocarbons. Argon (99.9999%) and hydrogen gas (99.9999%) were employed as carrier or make-up gases respectively. Solution 1H NMR, acquired with Varian NMRS 600 MHZ, was employed at the end of experiments to characterize liquid products. To be specific, 700 μL aliquot of the electrolyte was mixed with 70 μL of dimethyl sulfoxide (DMSO) standard solution (20 mM DMSO in D₂O).

### CONCLUSIONS

In conclusion, we report a hybrid catalyst with complementary single-atom Ni and nanoscale Cu active modules that permits cascading CO₂-to-CO and CO-to-C₂H₄ processes for the efficient production of C₂H₄. Compared with traditional single-component catalysts, this modular design presents a novel and facile strategy for cross-module transfer and enriching key intermediate *CO onto the Cu surface for the C—C coupling to the desired product, as validated by in situ SEIRAS. The resultant Cu/Ni-NAC hybrid catalyst with optimized two module compositions produces C₂H₄ with 50% FE at −1.6 V vs. RHE in 0.5 M KHCO₃ solution and with 66% FE at −0.5 V vs. RHE in 10 M KOH condition. Moreover, the hybrid catalyst is durable under flow cell conditions. The highlighted modular design of the eCO₂RR catalyst is likely applicable to other catalytic applications that involve multiple steps with key intermediate formation and utilization occurring at distinct units.

### REFERENCES

(1) Pörtner, H.-O.; Roberts, D. C.; Tignor, M.; Poloczanska, E. S.; Mintenbeck, K.; Alegría, A.; Craig, M.; Langsdorf, S.; Löschke, S.; Möller, V.; Okem, A.; Rama, B. Climate change 2022: Impacts, adaptation, and vulnerability. IPCC. 2022. 3056. doi: 10.1017/9781009325844.
(2) Centi, G.; Perathoner, S. Opportunities and Prospects in the Chemical Recycling of Carbon Dioxide to Fuels. Catal. Today 2009, 148, 191-205.
(3) Appel, A. M.; Bercaw, J. E.; Bocarsly, A. B.; Dobbek, H.; Dubois, D. L.; Dupuis, M.; Ferry, J. G.; Fujita, E.; Hille, R.; Kenis, P. J. A.; Kerfeld, C. A.; Morris, R. H.; Peden, C. H. F.; Portis, A. R.; Ragsdale, S. W.; Rauchfuss, T. B.; Reek, J. N. H.; Seefeldt, L. C.; Thauer, R. K.; Waldrop, G. L. Frontiers, Opportunities, and Challenges in Biochemical and Chemical Catalysis of CO2 Fixation. Chem. Rev. 2013, 113, 6621-6658.
(4) Chen, C.; Kotyk, J. F. K.; Sheehan, S. W. Progress toward Commercial Application of Electrochemical Carbon Dioxide Reduction. Chem 2018, 4, 2571-2586.
(5) Jin, S.; Hao, Z.; Zhang, K.; Yan, Z.; Chen, J. Advances and Challenges for the Electrochemical Reduction of CO2 to CO: From Fundamentals to Industrialization. Angew. Chem. Int. Ed. 2021, 60, 20627-20648.
(6) Wang, C.; Chen, J.; Ding, Y.; Cai, P.; Yi, L.; Li, Y.; Tu, C.; Hou, Y.; Wen, Z.; Dai, L. Electrocatalysis for CO2 Conversion: From Fundamentals to Value-Added Products. Chem. Soc. Rev. 2021, 50, 4993-5061.
(7) Ross, M. B.; De Luna, P.; Li, Y.; Dinh, C. T.; Kim, D.; Yang, P.; Sargent, E. H. Designing materials for electrochemical carbon dioxide recycling. Nat. Catal. 2019, 2 (8), pp. 648-658.
(8) Hori, Y.; Kikuchi, K.; Suzuki, S. Production of CO and CH4 in Electrochemical Reduction of CO2 at Metal-Electrodes in Aqueous Hydrogencarbonate Solution. Chem. Lett. 1985, 11, 1695-1698.
(9) Yin, Z.; Palmore, G. T. R.; Sun, S. Electrochemical Reduction of CO2 Catalyzed by Metal Nanocatalysts. Trends Chem. 2019, 1, 739-750.
(10) Todorova, T. K.; Schreiber, M. W.; Fontecave, M. Mechanistic Understanding of CO2 Reduction Reaction (CO2RR) Toward Multicarbon Products by Heterogeneous Copper-Based Catalysts. ACS Catal. 2020, 10, 1754-1768.
(11) Loiudice, A.; Lobaccaro, P.; Kamali, E. A.; Thao, T.; Huang, B. H.; Ager, J. W.; Buonsanti, R. Tailoring Copper Nanocrystals towards C2 Products in Electrochemical CO2 Reduction. Angew. Chem. Int. Ed. 2016, 55, 5789-5792.
(12) Huang, Y.; Handoko, A. D.; Hirunsit, P.; Yeo, B. S. Electrochemical Reduction of CO2 Using Copper Single-Crystal Surfaces: Effects of CO* Coverage on the Selective Formation of Ethylene. ACS Catal. 2017, 7, 1749-1756.
(13) Luo, T.; Liu, K.; Fu, J.; Chen, S.; Li, H.; Hu, J.; Liu, M. Tandem Catalysis on Adjacent Active Motifs of Copper Grain Boundary for Efficient CO2 Electroreduction toward C2 Products. J. Energy Chem. 2022, 70, 219-223.
(14) Choi, C.; Cheng, T.; Flores Espinosa, M.; Fei, H.; Duan, X.; Goddard 3rd, W. A.; Huang, Y. A Highly Active Star Decahedron Cu Nanocatalyst for Hydrocarbon Production at Low Overpotentials. Adv. Mater. 2018, e1805405.
(15) Li, Y.; Cui, F.; Ross, M. B.; Kim, D.; Sun, Y.; Yang, P. Structure-sensitive CO2 electroreduction to hydrocarbons on ultrathin 5-fold twinned copper nanowires. Nano Lett. 2017, 17 (2), pp. 1312-1317.
(16) Shang, L.; Lv, X.; Zhong, L.; Li, S.; Zheng, G. Efficient CO2 Electroreduction to Ethanol by Cu3Sn Catalyst. Small Methods 2022, 6, 2101334.
(17) Ma, S.; Sadakiyo, M.; Heima, M.; Luo, R.; Haasch, R. T.; Gold, J. I.; Yamauchi, M.; Kenis, P. J. Electroreduction of Carbon Dioxide to Hydrocarbons Using Bimetallic Cu—Pd Catalysts with Different Mixing Patterns. J. Am. Chem. Soc. 2017, 139, 47-50.
(18) Kibria, M. G.; Dinh, C. T.; Seifitokaldani, A.; De Luna, P.; Burdyny, T.; Quintero-Bermudez, R.; Ross, M. B.; Bushuyev, O. S.; García de Arquer, F. P.; Yang, P.; Sinton, D. A surface reconstruction route to high productivity and selectivity in CO2 electroreduction toward C2+ hydrocarbons. Adv. Mater. 2018, 30 (49), 1804867.
(19) De Luna, P.; Quintero-Bermudez, R.; Dinh, C. T.; Ross, M. B.; Bushuyev, O. S.; Todorović, P.; Regier, T.; Kelley, S. O.; Yang, P.; Sargent, E. H. Catalyst electro-redeposition controls morphology and oxidation state for selective carbon dioxide reduction. Nat. Catal. 2018, 1(2), 103-110.
(20) Merino-Garcia, I.; Albo, J.; Solla-Gullon, J.; Montiel, V.; Irabien, A. Cu oxide/ZnO-based surfaces for a selective ethylene production from gas-phase CO2 electroconversion. J. CO2 Util. 2019 31, 135-142.
(21) Chen, X.; Chen, J.; Alghoraibi, N. M.; Henckel, D. A.; Zhang, R.; Nwabara, U. O.; Madsen, K. E.; Kenis, P. J. A.; Zimmerman, S. C.; Gewirth, A. A. Electrochemical CO2-to-Ethylene Conversion on Polyamine-Incorporated Cu Electrodes. Nat. Catal. 2020, 4, 20-27.
(22) Santos-Lorenzo, J.; San José-Velado, R.; Albo, J.; Beobide, G.; Castano, P.; Castillo, O.; Luque, A.; Pérez-Yáñez, S.; A straightforward route to obtain zirconium based metal-organic gels. Micropor. Mesopor. Mat. 2019 284, 128-132.
(23) Albo, J.; Maite, P. I.; Garikoitz, B.; Angel I. Cu/Bi metal-organic framework-based systems for an enhanced electrochemical transformation of CO2 to alcohols. J. CO2 Util. 2019, 33, 157-165.
(24) Perfecto-Irigaray, M.; Albo, J.; Beobide, G.; Castillo, O.; Irabien, A.; Pérez-Yáñez, S. Synthesis of heterometallic metal-organic frameworks and their performance as electrocatalyst for CO 2 reduction. RSC Adv. 2018, 18, 21092-21099.
(25) Albo, J.; Vallejo, D.; Beobide, G.; Castillo, O.; Castaño, P.; Irabien, A.; Copper-based metal-Organic porous materials for CO2 electrocatalytic reduction to alcohols. ChemSusChem 2017, 6, 1100-1109.
(26) Dinh, C.-T.; Burdyny, T.; Kibria, M. G.; Seifitokaldani, A.; Gabardo, C. M.; Garcia de Arquer, F. P.; Kiani, A.; Edwards, J. P.; De Luna, P.; Bushuyev, O. S.; Zou, C.; Quintero-Bermudez, R.; Pang, Y.; Sinton, D.; Sargent, E. H. Carbon Dioxide Electroreduction to Ethylene via Hydroxide-Mediated Copper Catalysis at an Abrupt Interface. Science 2018, 360, 783-787.
(27) Salvatore, D.; Gabardo, C.; Reyes, A.; Salvatore, D. A.; Gabardo, C. M.; O, C. P.; Holdcroft, S.; Pintauro, P.; Bahar, B.; Hickner, M.; Bae, C.; Sinton, D.; Sargent, E. H.; Berlinguette, C. P. Designing Anion Exchange Membranes for CO2 Electrolysers. Nat. Energy. 2021, 6, 339-348.
(28) Mu, S.; Lu, H.; Wu, Q.; Li, L.; Zhao, R.; Long, C.; Cui, C. Hydroxyl radicals dominate reoxidation of oxide-derived Cu in electrochemical CO2 reduction. Nat. Commun. 2022, 13, 1-8.
(29) Lei, Q.; Zhu, H.; Song, K.; Wei, N.; Liu, L.; Zhang, D.; Yin, J.; Dong, X.; Yao, K.; Wang, N.; Li, X. Investigating the origin of enhanced C2+ selectivity in oxide-/hydroxide-derived copper electrodes during CO₂ electroreduction. J. Am. Chem. Soc. 2022, 142, 4213-4222.
(30) Jeong, H. M.; Kwon, Y.; Won, J. H.; Lum, Y.; Cheng, M. J.; Kim, K. H.; Head-Gordon, M.; Kang, J. K. Atomic-Scale Spacing between Copper Facets for the Electrochemical Reduction of Carbon Dioxide. Adv. Energy Mater. 2020, 10, 1903423.
(31) Varandili, S. B.; Huang, J.; Oveisi, E.; De Gregorio, G. L.; Mensi, M.; Strach, M.; Vavra, J.; Gadiyar, C.; Bhowmik, A.; Buonsanti, R. Synthesis of Cu/CeO2-x nanocrystalline heterodimers with interfacial active sites to promote CO2 electroreduction. ACS Catal. 2019, 9, 5035-5046.
(32) Zhou, Y.; Che, F.; Liu, M.; Zou, C.; Liang, Z.; De Luna, P.; Yuan, H.; Li, J.; Wang, Z.; Xie, H.; Li, H. Dopant-induced electron localization drives CO2 reduction to C2 hydrocarbons. Nat. Chem. 2018, 10, 974-980.
(33) Su, X.; Jiang, Z.; Zhou, J.; Liu, H.; Zhou, D.; Shang, H.; Ni, X.; Peng, Z.; Yang, F.; Chen, W.; Qi, Z. Complementary Operando Spectroscopy identification of in-situ generated metastable charge-asymmetry Cu2-CuN3 clusters for CO₂ reduction to ethanol. Nat. Commun. 2022, 13, 1-11.
(34) Rossi, K.; Buonsanti, R. Shaping Copper Nanocatalysts to Steer Selectivity in the Electrochemical CO2 Reduction Reaction. Acc. Chem. Res. 2022, 55, 629-637.
(35) Xiao, C.; Zhang, J. Architectural Design for Enhanced C2 Product Selectivity in Electrochemical CO2 Reduction Using Cu-Based Catalysts: A Review. ACS Nano 2021, 15, 7975-8000.
(36) Suo, H.; Solan, G. A.; Ma, Y.; Sun, W. H. Developments in Compartmentalized Bimetallic Transition Metal Ethylene Polymerization Catalysts. Coord. Chem. Rev. 2018, 372, 101-116.
(37) Zhang, H.; Zhang, Y.; Li, Y.; Ahn, S.; Palmore, G. T. R.; Fu, J.; Peterson, A. A.; Sun, S. Cu Nanowire-Catalyzed Electrochemical Reduction of CO or CO2. Nanoscale 2019, 11, 12075-12079.
(38) Wang, L.; Nitopi, S. A.; Bertheussen, E.; Orazov, M.; Morales-Guio, C. G.; Liu, X. Y.; Higgins, D. C.; Chan, K. R.; Norskov, J. K.; Hahn, C.; Jaramillo, T. F. Electrochemical Carbon Monoxide Reduction on Polycrystalline Copper: Effects of Potential, Pressure, and PH on Selectivity toward Multicarbon and Oxygenated Products. ACS Catal. 2018, 8, 7445-7454.
(39) An, H.; Wu, L.; Mandemaker, L. D. B.; Yang, S.; de Ruiter, J.; Wijten, J. H. J.; Janssens, J. C. L.; Hartman, T.; van der Stam, W.; Weckhuysen, B. M. Sub-Second Time-Resolved Surface-Enhanced Raman Spectroscopy Reveals Dynamic CO Intermediates during Electrochemical CO2 Reduction on Copper. Angew. Chem. Int. Ed. 2021, 60, 16576-16584.
(40) Raciti, D.; Cao, L.; Livi, K. J. T.; Rottmann, P. F.; Tang, X.; Li, C.; Hicks, Z.; Bowen, K. H.; Hemker, K. J.; Mueller, T.; Wang, C. Low-Overpotential Electroreduction of Carbon Monoxide Using Copper Nanowires. ACS Catal. 2017, 7, 4467-4472.
(41) Nitopi, S.; Bertheussen, E.; Scott, S. B.; Liu, X.; Engstfeld, A. K.; Horch, S.; Seger, B.; Stephens, I. E. L.; Chan, K.; Hahn, C.; Nørskov, J. K.; Jaramillo, T. F.; Chorkendorff, I. Progress and Perspectives of Electrochemical CO2 Reduction on Copper in Aqueous Electrolyte. Chem. Rev. 2019, 119, 7610-7672.
(42) Luo, Z.; Yin, Z.; Yu, J.; Yan, Y.; Hu, B.; Nie, R.; Kolln, A. F.; Wu, X.; Behera, R. K.; Chen, M.; Zhou, L.; Liu, F.; Wang, B.; Huang, W.; Zhang, S.; Qi, L. General Synthetic Strategy to Ordered Mesoporous Carbon Catalysts with Single-Atom Metal Sites for Electrochemical CO2 Reduction. Small 2022, 2107799.
(43) Luo, Z.; Nie, R.; Nguyen, V. T.; Biswas, A.; Behera, R. K.; Wu, X.; Kobayashi, T.; Sadow, A.; Wang, B.; Huang, W.; Qi, L. Transition Metal-like Carbocatalyst. Nat. Commun. 2020, 11, 1-9.
(44) Grosse, P.; Gao, D.; Scholten, F.; Sinev, I.; Mistry, H.; Roldan Cuenya, B. Dynamic Changes in the Structure, Chemical State and Catalytic Selectivity of Cu Nanocubes during CO₂ Electroreduction: Size and Support Effects. Angew. Chem. Int. Ed. 2018, 57, 6192-6197.
(45) Mukherjee, S.; Yang, X.; Shan, W.; Samarakoon, W.; Karakalos, S.; Cullen, D. A.; More, K.; Wang, M.; Feng, Z.; Wang, G.; Wu, G. Atomically Dispersed Single Ni Site Catalysts for Nitrogen Reduction toward Electrochemical Ammonia Synthesis Using N2 and H2O. Small Methods 2020, 4, 1900821.
(46) Dunwell, M.; Lu, Q.; Heyes, J. M.; Rosen, J.; Chen, J. G.; Yan, Y.; Jiao, F.; Xu, B. The Central Role of Bicarbonate in the Electrochemical Reduction of Carbon Dioxide on Gold. J. Am. Chem. Soc. 2017, 139, 3774-3783.
(47) Gunathunge, C. M.; Ovalle, V. J.; Li, Y.; Janik, M. J.; Waegele, M. M. Existence of an Electrochemically Inert CO Population on Cu Electrodes in Alkaline PH. ACS Catal. 2018, 8, 7507-7516.
(48) Lee, J. H.; Kattel, S.; Jiang, Z.; Xie, Z.; Yao, S.; Tackett, B. M.; Xu, W.; Marinkovic, N. S.; Chen, J. G., Tuning the activity and selectivity of electroreduction of CO2 to synthesis gas using bimetallic catalysts. Nat. Commun. 2019, 10, 1-8.

TABLE S1
CO₂RR performance comparison among recent Cu-based catalysts for C₂H₄ production.

| | | | | Current | |
|---|---|---|---|---|---|
| | | | C₂H₄FE | density | |
| Catalyst | Electrolyte | Potential (V) | (%) | (mA · cm⁻²) | Refs |
| Cu/Ni-NAC | 10M KOH | −0.5 | 66 | 100 | This work |
| Cu oxides/ZnO | 0.1M | −1.8 | 91.1 | 7.5 | J. CO₂ Util. 2019, 31, 135- |
| | KHCO₃ | | | | 142. |
| Cu cube | 0.1M | −1.1 | 41 | 5.7 | Angew. Chem. Int. Ed. |
| | KHCO₃ | | | | 2016, 55, 5789-5792. |
| Cu cube | 10M KOH | −0.47 | 60 | 240 | Nano Lett. 2019, 19, 8461- |
| | | | | | 8458 |
| CuAg film | 1M KOH | −0.7 | 60 | 300 | J. Am. Chem. Soc. 2018, |
| | | | | | 140, 5791-5797 |
| Star decahedron Cu | 0.1M | −0.99 | 52.43 | 33 | Adv. Mater. 2018, 31, |
| | KHCO₃ | | | | e1805405. |
| Boron-doped Cu | 0.1M | −1.0 | 53 | 70 | Nat. Chem. 2018, 10, |
| | KHCO₃ | | | | 974-980. |
| Cu nanowires with | 0.1M | −1.0 | 77 | 23 | Nat. Catal. 2020, 3, |
| surface steps | KHCO₃ | | | | 804-812. |
| Cu | 10M KOH | −0.54 | 66 | 275 | Science 2018, 360, 783- |
| | | | | | 787. |
| Cu with ionic liquid | 0.1M | −1.49 | 77.3 | 19.7 | Angew. Chem. Int. Ed. |
| | KHCO₃ | | | | 2022, 61, e202200039 |
| polyamine- | 10M KOH | −0.47 | 87 | 433 | Nat. Catal. 2020, 4, 20- |
| incorporated Cu | | | | | 27. |
| MOF with Bi—Cu | 0.1M | −1.2 | 42 | 5.2 | ACS Catal. 2022, 12, |
| site | KHCO₃ | | | | 7986-7993 |

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A hybrid catalyst comprising a plurality of Cu nanowires and a single-atom nickel on nitrogen assembly carbon (Ni-NAC).

2. The catalyst of claim 1, wherein the Cu nanowire has a diameter of about 10 to 100 nanometer and a length of about 0.5 to 50 μm.

3. The catalyst of claim 2, wherein the Cu nanowires have a dominant {100} surface facets.

4. The catalyst of claim 2, wherein the Cu nanowires have a face-centered cubic metallic structure.

5. The catalyst of claim 1, wherein the Ni-NAC is a nitrogen-doped ordered mesoporous carbon embedded with single-atom nickel.

6. The catalyst of claim 1, wherein the Ni-NAC has a Ni loading of about 1.5 to 3 weight %.

7. The catalyst of claim 1, wherein ratio of Cu nanowires:Ni-NAC of about 2:1 to 20:1.

8. The catalyst of claim 1, wherein the Ni-NAC does not include a Ni nanoparticle, wherein the Ni present in the Ni-NAC consists of single-atom Ni.

9. The catalyst of claim 1, further comprising powered carbon black mixed with the Cu nanowires and Ni-NAC.

10. The catalyst of claim 9, wherein ratio of Cu nanowires:Ni-NAC:powered carbon black of about 2:1:1 to 20:1:9.

11. The catalyst of claim 1, wherein the Cu nanowire has a diameter of 10 to 100 nanometer and a length of 0.5 to 50 μm, wherein the Cu nanowires have a dominant {100} surface facets, wherein the Ni-NAC is a nitrogen-doped ordered mesoporous carbon embedded with single-atom nickel, wherein the Ni-NAC has a Ni loading of about 1.5 to 3 weight %, wherein ratio of Cu nanowires:Ni-NAC of about 2:1 to 20:1, wherein the Ni-NAC does not include a Ni nanoparticle, wherein the Ni present in the Ni-NAC consists of single-atom Ni.

12. The catalyst of claim 11, wherein ratio of Cu nanowires:Ni-NAC:powered carbon black of about 2:1:1 to 20:1:9.

13. The catalyst of claim 1, wherein the Cu nanowire has a diameter of 10 to 100 nanometer and a length of 0.5 to 50 μm, wherein the Cu nanowires have a face-centered cubic metallic structure, wherein the Ni-NAC is a nitrogen-doped ordered mesoporous carbon embedded with single-atom nickel, wherein the Ni-NAC has a Ni loading of about 1.5 to 3 weight %, wherein ratio of Cu nanowires:Ni-NAC of about 2:1 to 20:1, wherein the Ni-NAC does not include a Ni nanoparticle, wherein the Ni present in the Ni-NAC consists of single-atom Ni.

14. The catalyst of claim 13, wherein ratio of Cu nanowires:Ni-NAC:powered carbon black of about 2:1:1 to 20:1:9.

15. The catalyst of claim 1, wherein the Cu nanowire has a diameter of 10 to 100 nanometer and a length of 0.5 to 50 μm, wherein the Ni-NAC is a nitrogen-doped ordered mesoporous carbon embedded with single-atom nickel, wherein the Ni-NAC has a Ni loading of about 1.5 to 3 weight %, wherein ratio of Cu nanowires:Ni-NAC of about 2:1 to 20:1, wherein the Ni-NAC does not include a Ni nanoparticle, wherein the Ni present in the Ni-NAC consists of single-atom Ni.

16. A method of converting CO₂ to ethylene comprising:
exposing CO₂ to a hybrid catalyst as described in claim 1; and
forming ethylene.

17. The method of claim 16, wherein forming ethylene comprises producing ethylene with about 50% Faradaic efficiency (FE) at −1.6 V vs. RHE in 0.5 M KHCO₃ solution and with about 66% FE at −0.5 V vs. RHE in 10 M KOH condition.

18. The method of claim 16, wherein the Cu nanowire has a diameter of about 10 to 100 nanometer and a length of about 0.5 to 50 μm, wherein the Ni-NAC is a nitrogen-doped ordered mesoporous carbon embedded with single-atom nickel, wherein the Ni-NAC has a Ni loading of about 1.5 to 3 weight %, wherein ratio of Cu nanowires:Ni-NAC of about 2:1 to 20:1, wherein the Ni-NAC does not include a Ni nanoparticle, wherein the Ni present in the Ni-NAC consists of single-atom Ni.

19. A system, comprising:
a hybrid catalyst as described in claim 1,
an introduction system to expose CO₂ to the hybrid catalyst, wherein upon introduction of the CO₂ to the hybrid catalyst ethylene is produced.

20. The system of claim 19, wherein the Cu nanowire has a diameter of about 10 to 100 nanometer and a length of about 0.5 to 50 μm, wherein the Ni-NAC is a nitrogen-doped ordered mesoporous carbon embedded with single-atom nickel, wherein the Ni-NAC has a Ni loading of about 1.5 to 3 weight %, wherein ratio of Cu nanowires:Ni-NAC of about 2:1 to 20:1, wherein the Ni-NAC does not include a Ni nanoparticle, wherein the Ni present in the Ni-NAC consists of single-atom Ni.
